(19) United States
(12) Patent Application Publication
(10) Pub. No.: US 2013/0158111 A1
(43) Pub. Date: Jun. 20, 2013

(54) Compositions and Methods for Depleting Mutant p53

(71) Applicant: Georgetown University, Washington, DC (US)

(72) Inventors: Fung-Lung Chung, Rockville, MD (US); Anthony Di Pasqua, Carrboro, NC (US)

(73) Assignee: Georgetown University, Washington, DC (US)

(21) Appl. No.: 13/721,681

(22) Filed: Dec. 20, 2012

Related U.S. Application Data
(60) Provisional application No. 61/578,023, filed on Dec. 20, 2011.

(51) Int. Cl. A61K 31/26; A61K 45/00

(52) U.S. Cl. CPC A61K 31/26; A61K 45/00
USPC 514/515; 514/514

(57) ABSTRACT

The invention is directed towards methods of treating abnormal tissues in a subject or sensitizing an abnormal tissue to a chemotherapeutic agent. The methods comprise screening at least a portion of an abnormal tissue for at least one mutant form of p53 protein, and administering at least one isothiocyanate (ITC) to the subject or abnormal tissue if the abnormal tissue is positive for the at least one mutant form of p53.

### STATEMENT REGARDING FEDERALLY SPONSORED RESEARCH OR DEVELOPMENT

Part of the work performed during development of this invention utilized U.S. Government funds under National Institutes of Cancer Grant No. CA100583. The U.S. Government has certain rights in this invention.

### BACKGROUND OF THE INVENTION

1. Field of the Invention

The invention is directed towards methods of treating abnormal tissues in a subject or sensitizing an abnormal tissue to a chemotherapeutic agent. The methods comprise screening at least a portion of an abnormal tissue for at least one mutant form of p53 protein, and administering at least one isothiocyanate (ITC) to the subject or abnormal tissue if the abnormal tissue is positive for the at least one mutant form of p53.

2. Background of the Invention

The p53 protein, encoded by the TP53 gene, is an important tumor suppressor. Under normal conditions, cells do not contain high levels of this protein. If a healthy cell is damaged, p53 is normally expressed and is followed by inhibition of cell growth or programmed cell death (apoptosis). To induce apoptosis, p53 must bind to a specific DNA sequence. Numerous studies have demonstrated that p53 plays a critical role in directing a cell to stop growing and/or to undergo apoptosis. p53 has thus become recognized as one of the most important cellular guardians, preventing damaged cells from developing into an abnormal such as a tumor. In fact, approximately 50% of human tumor possess some form of mutant p53, which, in turn is unable to bind target DNA sequences thus allowing an unregulated growth and division of such tumor cells. Indeed, mutation of p53 is considered the most frequent genetic alteration occurring in human cancer. Further, tumors associated with mutant p53 are often more resistant to chemotherapy than tumors with wild-type p53.

In tumor cells, mutant forms of p53 are commonly present at elevated levels compared to wild-type p53 in healthy cells. The fact that wild-type p53 is still present even in tumor cell offers the possibility of somehow eliminating the mutant forms of p53 to allow the cell the ability regain wild-type p53 function such as the ability to bind DNA, the tumor suppressor. This imbalance also offers the possibility of selectively killing cancer cells over healthy cells via reactivation of mutant p53, which could lead to medications without the devastating side effects often associated with conventional chemotherapy.

### SUMMARY OF THE INVENTION

The invention is directed towards methods of treating abnormal tissues in a subject in need of treatment. The methods comprise screening at least a portion of an abnormal tissue for at least one mutant form of p53 protein, and administering at least one isothiocyanate (ITC) to the subject if the abnormal tissue is positive for the at least one mutant form of p53.

The invention is also directed towards methods of sensitizing an abnormal tissue to a chemotherapeutic agent. The methods comprise screening at least a portion of an abnormal tissue for at least one mutant form of p53 protein, and administering at least one isothiocyanate (ITC) to the abnormal tissue if the abnormal tissue is positive for the at least one mutant form of p53.

The invention also provides for methods of altering the three-dimensional conformation of a mutant p53 protein, with the methods comprising administering at least one isothiocyanate (ITC) to the mutant p53 protein to alter the three-dimensional conformation of the mutant p53 protein.

### BRIEF DESCRIPTION OF THE DRAWINGS

FIGS. 1-5 and their descriptions are reprinted with permission from “Selective Depletion of Mutant p53 by Cancer Chemopreventive Isothiocyanates and Their Structure—Activity Relationships,” Xiantao Wang, et al., J. Med. Chem. Copyright (2011) American Chemical Society.”

FIG. 1 depicts PEITC causing a rapid depletion of mutant p53 in H596 cells. (A) PEITC decreased mutant p53 protein in a time- and dose-dependent manner. H596 cells were treated with 15 μM PEITC for various times or incubated with various concentrations of PEITC for 2 h. p53 and β-actin levels were determined by immunoblotting. Lower panel, H596 cells were treated with 15 μM PEITC for 2, 4, and 6 hours, mRNA was then isolated, and RT-PCR for p53 and β-actin mRNA was performed. (B) Structures of ITCs and NMPEA, which is an analog of PEITC without the ITC functional group and was used as a negative control. (C) Effects of PEITC, BITC, SFN, and AITC on mutant p53 protein in H596 cells. (D) NMPEA has no effect on p53 mutant protein in H596 cells.

FIG. 2 depicts PEITC depleting p53 protein in human cancer cells with different p53 mutation but not in cells with wild-type p53. (A) Cell lines harboring mutant p53 were treated with 15 μM PEITC (MDA-MB-231, MDA-MB-468, and SCC-4) or 10 μM PEITC (DU145 and SW480) for the indicated times. (B) Cells with wild-type p53 were treated with 20 μM PEITC (HCT116, A549, and MCF-7) or 15 μM PEITC (MCF-10A) for the indicated times. (C) H1299-143A cells with p53 mutant conformation (at 37° C.) are more sensitive to PEITC-induced depletion than those with wild-type conformation (at 32.5° C.). H1299-175H cells are used as control. Both cells were treated with 20 μM PEITC for 24 h.

FIG. 3 depicts the structure-activity relationships for the depletion of mutant p53 by ITCs. (A) Structures of the naturally occurring and synthetic ITCs used. (B, C) Depletion of mutant p53 protein by ITCs in H596 non-small-cell lung cancer cells treated with 20 μM ITCs, as determined by immunoblotting. (D, E) MDA-MB-231 breast cancer cells were treated with 10 μM ITCs.

FIG. 4 depicts cysteine binding in mutant (G245C) p53 DNA-binding domain by ITCs and subsequent conformational changes. (A) Cysteine binding in the mutant (G245C) p53 DNA-binding domain by DMSO (control), ITCs, or iodoacetamide, measured using a monochlorobimane fluorometric assay (λₑₓ 380 nm; λₑₘ 485 nm). (B) Intrinsic fluorescence emission spectra of mutant (G245C) p53 DNA-binding domain at 32° C. (black solid line) incubated with, from highest intensity to lowest, DMSO (black large-dashed line), SFN (black small-dashed line), D-Rα-methylbenzyl ITC (dark gray line), BITC (medium gray line), or 4-phenoxybenzyl ITC (light gray line).

FIG. 5 depicts cells with mutant p53 being more sensitive to PEITC-induced apoptosis than wild-type cells, and induction of apoptosis by various ITCs in a cell line with mutant p53. (A) A549 (wt p53) and H596 (mutant p53) non-small-cell lung cancer cells, MCF-7 (wt p53) and MDA-MB-231 and MDA-MB-468 (mutant p53) breast cancer cells, and HCT116 (wt p53) and SW480 (mutant p53) colon cancer cells were treated with DMSO and PEITC for 24 h. Caspase-3 activity was analyzed by a caspase-3 colorimetric assay kit. The data represents the mean (SD of three independent experiments (*P<0.01). (B) Apoptosis in MDA-MB-231 breast cancer cells treated with 10 μM ITCs measured using FITC-annexin V and propidium iodide. Shown are cells in early apoptosis (FITC-annexin V±/Pl−); *p<0.05 compared with DMSO (control), **p<0.05 compared with BITC.

FIG. 6 depicts the comparison of tumor volume of LNCap (A) and DU145 (B) cells in mice fed with diet with and without PEITC for 8 weeks.

FIG. 7 depicts the expression level of mutant p53 in Du145 tumors in mice with control and PEITC diets.

FIG. 8 depicts the average expression level of p53 in DU145 tumors.

FIG. 9 depicts Effect of PEITC exposure on the growth of tumor cell lines expressing mutant or WT p53.

FIG. 10 depicts the effect of PEITC treatment on apoptosis and cell cycle progression on mutant p53 expressing tumor cell lines.

FIG. 11 depicts the conformational change in mutant p53—R175H protein upon PEITC exposure.

### DETAILED DESCRIPTION OF THE INVENTION

The invention is directed towards methods of treating abnormal tissue in a subject. The methods comprise screening at least a portion of an abnormal tissue for at least one mutant form of p53 protein, and administering at least one isothiocyanate (ITC) to the subject or abnormal tissue if the abnormal tissue is positive for the at least one mutant form of p53.

The invention also provides for methods of altering the three-dimensional conformation of a mutant p53 protein, with the methods comprising administering at least one isothiocyanate (ITC) to the mutant p53 protein to alter the three-dimensional conformation of the mutant p53 protein. In one embodiment, the mutant p53 protein of which the conformation is to be altered is present in a cell or group of cells. In another embodiment, the cell or group of cells containing the mutant p53 protein of which the conformation is to be altered is comprised within a subject, such as, but not limited to a mammal.

The methods of the present are for subjects in need of treatment of an abnormal tissue. As used herein, the terms “subject” and “patient” are used interchangeably. In one embodiment, the subject is a mammal, such as a dog, cat, horse, cow, pig, mouse, rat, non-human primate and human. In one specific embodiment, the subject is a human subject.

The methods are directed to treatment of those subjects in need of treatment. As used herein, “in need of treatment” indicates that the subject has been diagnosed with an abnormal growth or tissue, or that the subject is suspected of having an abnormal growth or tissue. As used herein, “abnormal growth” or “abnormal tissue” can be used interchangeably. An abnormal tissue for the purposes of this disclosure is used to mean a portion of a tissue that is pathologically distinct from the portion of the tissue that is deemed “normal” or healthy tissue. The pathologically distinct tissue may or may not be metabolically “normal” compared to the healthy, normal tissue. An abnormal tissue, as used herein, also means an abnormal cell proliferation that that is not normally present in healthy tissue. Abnormal tissue thus includes, but is not limited to, neoplasias, hyperplasias, benign tumors and malignant tumors when referring to solid tissue or organs. Abnormal tissue that can be treated using the methods of the present invention include such tissue deriving from, but not limited to, lung tissue, bronchial tissue, tracheal tissue, laryngeal tissue, heart tissue, breast tissue, lymphatic tissue, esophageal tissue, stomach tissue, intestinal tissue, colon tissue, bladder tissue, pancreatic tissue, liver tissue, kidney tissue, prostate tissue, ovarian tissue, cervical tissue, uterine tissue, muscular tissue, skin, squamous cell tissue, brain tissue and bone tissue, to name a few. Abnormal growth or abnormal tissue is also used in conjunction with abnormal cells and components of the blood, immune and lymph systems, such that abnormal tissue, as used herein, includes but is not limited to abnormal red blood cells, white blood cells, platelets, B cells, T cells and the like.

In various embodiments, “treatment” or “treating” refers to an amelioration of a disease, disorder or abnormal growth, or at least one discernible symptom thereof. In another embodiment, “treatment” or “treating” refers to an amelioration of at least one measurable physical parameter not necessarily discernible by the patient. In yet another embodiment, “treatment” or “treating” refers to inhibiting the progression of a disease, disorder or abnormal growth, either physically, e.g., stabilization of a discernible symptom, physiologically, e.g., stabilization of a physical parameter, or both. In yet another embodiment, “treatment” or “treating” refers to delaying the onset of a disease, disorder or abnormal growth.

In certain embodiments, the methods the invention are directed to administering at least one isothiocyanate (ITC) to the subject or abnormal tissue as a preventative measure against the acquisition of an abnormal growth. As used herein, “prevention” or “preventing” refers to a reduction of the risk of acquiring a given disease, disorder or abnormal growth. In one embodiment, the compositions of the present invention are administered as a preventative measure to a patient having a genetic predisposition to abnormal cell proliferation or an abnormal growth, e.g., a subject that tests positive for a BRCA1 and/or BRCA2 mutation or patient with Li-Fraumeni syndrome that possess p53 mutations.

The methods of the present invention comprise screening at least a portion of an abnormal tissue from a subject for at least one mutant form of p53 protein. In one embodiment, normal tissue is also screened for the presence of at least one mutant form of p53 protein. The normal tissue may be screened in addition to the screening of the abnormal tissue, or the normal tissue may be screened, for example, after the abnormal tissue has been removed or excised from the patient.

In one embodiment, the tissue is screened for a mutant p53 protein, wherein the mutant p53 is a mutant of a wild-type p53. In a more specific embodiment, the mutant p53 is a mutant of the wild-type amino acid sequence human p53 as disclosed in SEQ ID NO:1 below, which is accessible through the UniProt Database as record number P04637, the entire record of which is incorporated by reference. The UniProt database is available on the world-wide-web at www.uniprot.org. In additional embodiments, the mutant p53 is a mutant of another organism's p53, depending on the species of the subject. The mutant p53 would, of course, correspond to a mutant of the p53 for that specific organism, e.g., monkey, dog, horse p53, etc.

| (SEQ ID NO: 1) | |
|---|---|
| 1 | MEEPQSDPSV EPPLSQETFS DLWKLLPENN VLSPLPSQAM DDLMLSPDDI EQWFTEDPGP |
| 61 | DEAPRMPEAA PPVAPAPAAP TPAAPAPAPS WPLSSSVPSQ KTYQGSYGFR LGFLHSGTAK |
| 121 | SVTCTYSPAL NKMFCQLAKT CPVQLWVDST PPPGTRVRAM AIYKQSQHMT EVVRRCPHHE |
| 181 | RCSDSDGLAP PQHLIRVEGN LRVEYLDDRN TFRHSVVVPY EPPEVGSDCT TIHYNYMCNS |
| 241 | SCMGGMNRRP ILTIITLEDS SGNLLGRNSF EVRVCACPGR DRRTEEENLR KKGEPHHELP |
| 301 | PGSTKRALPN NTSSSPQPKK KPLDGEYFTL QIRGRERFEM FRELNEALEL KDAQAGKEPG |
| 361 | GSRAHSSHLK SKKGQSTSRH KKLMFKTEGP DSD |

Record number P04637 in the UniProt database lists many mutations to the amino acid sequence of p53, all of which are incorporated by reference. In specific embodiments, the mutant p53 contains at least one mutation selected from the group of V143, P151, R175, G245, R248, R249, R273, P274, R280 and R282. In more specific embodiments, the mutant p53 has at least one mutation selected from V143A, P151S, R175H, P223L, M237I, G245C, G245H, G245S, R273H, P274L and R280K and or any combination thereof. Additional mutations are disclosed in Bai, L. and Zhu, W., J. Cancer Mol., 2(4):141-153 (2006), which is incorporated by reference. In one specific embodiment, the mutant p53 has at least one mutation in its N-terminal domain (approximately amino acids 1-42). In another specific embodiment, the mutant p53 has at least one mutation in its activation domain (approximately amino acids 43-63). In another specific embodiment, the mutant p53 has at least one mutation in its protein rich domain (approximately amino acids 64-92). In another specific embodiment, the mutant p53 has at least one mutation in its DNA-binding domain (approximately amino acids 100-300). In another specific embodiment, the mutant p53 has at least one mutation in its nuclear localization signaling domain (approximately amino acids 316-325). In another specific embodiment, the mutant p53 has at least one mutation in its homo-oligomerisation domain (approximately amino acids 307-355). In another specific embodiment, the mutant p53 has at least one mutation in its C-terminal domain (approximately amino acids 356-393). In another specific embodiment, the mutant p53 has one or more mutations in one or more of the domains listed herein.

Determining the sequence or putative sequence of p53 from tissue is a routine method in the art. For example, mRNA can be extracted from the cells of tissue and then converted to DNA by way of an RT-PCR reaction. The DNA can then be sequenced and the amino acid sequence correlating to the extracted mRNA can be deduced. Alternatively, genomic DNA can be extracted from the cells of tissue and sequenced directly. Other methods for detecting mutations include but are not limited to single-strand conformation polymorphism (SSCP) assay, denaturing gel electrophoresis (DGGE). Other methods of detecting mutant p53 include microarray assays such as but not limited to the p53 GeneChip™ Assay available from Affymetrix, Inc. (Santa Clara, Calif., USA). Yet another method for determining the presence of a mutant p53 is disclosed in U.S. Pat. No. 7,267,955, which is incorporated by reference.

The invention, however, is not limited by the specific identity of the mutant p53. Indeed, the methods described herein can be applied to any mutant p53. In one embodiment, the mutant p53 is a functional mutant, where the amino acid sequence or corresponding DNA sequence of the mutant p53 may or may not be known. Normally, p53 functions act as a tumor suppressor protein in a cell to, for example, activate DNA repair proteins, induce or promote arrest of cell division and/or induce or promote apoptosis. Accordingly, a functionally mutant P53 would manifest itself in cells that exhibit a loss of any one of these functions, such as but not limited to, loss of ability to activate DNA repair proteins, loss of ability to induce or promote arrest of cell division and/or loss of ability to induce or promote apoptosis. Thus, if the p53 isolated from the tissue of the patient exhibits an abnormal function, the p53 would be considered mutant for the purposes of the invention. Additionally, a functionally mutant P53 would manifest itself in cells that exhibit increased growth rates.

Assessing the functional activity of p53, whether normal or mutant, is routine in the art and includes such methods as those disclosed in Flaman, J., et al., Proc. Nat'l. Acad. Sci., 92:3963-3967 (1995) and Camplejohn, R. and Rutherford, J., Cell Prolif., 34:1-14 (2001), both of which are incorporated by reference. Other ways to assess the presence of a mutant p53 without sequencing include but are not limited to detecting the loss of expression of the p53 gene, such as detecting the loss of mRNA and/or p53 protein.

The methods comprise screening the abnormal tissue for mutant p53 prior to the administration of at least one isothiocyanate (ITC) to the subject. As used herein, the term “screening” or “screen” simply means testing for the presence or absence of a mutant form of p53, as described herein, in at least a portion of abnormal tissue or tissue suspected of being abnormal. The screening methods may, but need not, include screening tissue suspected of being normal or suspected of having normal/wild-type p53 as a comparison. In one embodiment, the screening methods comprise comparing the test results from the abnormal tissue or tissue suspected of being abnormal with a control. The control, as used herein, is normal, functional p53. If the screening methods rely on sequence analysis, the control would be the sequence of the normal or wild-type p53. Likewise, if the screening methods rely on functional data, the control would be the function of a normal, wild-type p53.

Because the sequence and function of p53 are so well known in the art, the methods described herein may rely on art-established information to establish a control for comparison of the test results to normal, wild-type p53. Alternatively, the screening methods may rely on the structural or functional status of p53 in normal tissue taken from the subject to establish a control for comparison purposes. Once a control is established, the data on the p53 from the abnormal tissue or suspected abnormal tissue is collected and compared to the control values. A change in p53 status, whether functional or structural, as compared to control values would indicate that the test p53 would be considered a mutant p53 for the purposes of this invention. In general, a loss or decrease of p53 function would indicate that the p53 is a mutant p53. In one embodiment, the functional change compared to control is a complete loss of p53 function. In another embodiment, the functional change compared to control is a partial loss of p53. As used herein “a partial loss of p53 function” means that the cells are capable of exhibiting some level of function of normal p53, but to a lesser or slower extent. For example, a partial loss of p53 function may mean that the cells become arrested in cell division to a lesser or slower extent.

In another embodiment, mutant p53 can be detected in other samples that are not necessarily adjacent to or directly from the abnormal tissue, including but not limited to such samples as serum, stool, or other body fluids, such as urine and sputum. The same techniques discussed above for detection of mutant p53 in tissues can be applied to other samples from a subject. By screening such samples, a simple early diagnosis can be achieved for many types of abnormal growths.

Once the abnormal or suspected abnormal tissue is screened and it is determined that the p53 is a mutant p53, at least one isothiocyanate (ITC) is administered to the subject. As used herein, the term “administer” and “administering” are used to mean introducing at least one compound into a subject. When administration is for the purpose of treatment, the substance is provided at, or after the diagnosis of an abnormal tissue or growth. The therapeutic administration of the compounds of the present invention serves to attenuate any symptom, or prevent additional symptoms from arising. When administration is for the purposes of preventing an abnormal growth or tissue from arising (“prophylactic administration”), the substance is provided in advance of any visible or detectable symptom. The prophylactic administration of the substance serves to attenuate subsequently arising symptoms or prevent symptoms from arising altogether. For example, after tissue biopsy the compounds of the present invention can be administered to the subject after all abnormal growth or tissues have been removed to reduce the likelihood of a recurrence or to reduce the likelihood of a new occurrence of developing an abnormal growth or tissue. In addition, the ITC compounds of the present invention may be administered prophylactically after a mutant p53 has been detected in a subject even though an abnormal tissue or growth has not been detected. The route of administration of the compound includes, but is not limited to, topical, transdermal, intranasal, vaginal, rectal, oral, subcutaneous intravenous, intraarterial, intramuscular, intraosseous, intraperitoneal, epidural and intrathecal.

As used herein, “isothiocyanate” (ITC) refers to any compound having the formula, R—N═C═S, where R may be saturated or unsaturated, substituted or unsubstituted, or an aliphatic or aromatic group. Non-limiting examples of R include phenethyl, benzyl, methyl; ethyl; propyl; isopropyl; n-butyl; t-butyl; s-butyl; pentyl; hexyl; heptyl; octyl; nonyl; decyl; undecane; phenyl; o-tolyl; 2-fluorophenyl; 3-fluorophenyl; 4-fluorophenyl; 2-nitrophenyl; 3-nitrophenyl; 4-nitrophenyl; 2-chlorophenyl; 2-bromophenyl; 3-chlorophenyl; 3-bromophenyl; 4-chlorophenyl; 2,4-dichlorophenyl; R-(+)-alpha-methylbenzyl; S-(−)-alpha-methylbenzyl; 3-isoprenyl-alpha, alpha-dimethylbenzyl; trans-2-phenylcyclopropyl; (SCN)CH₂C₆H₄CH₂—; (SCN)CH(CH₃)CH₂—C₆H₄CH₂CH(CH₃)CH₂—; CH₃S(O)CH₂CH₂CH₂CH₂—; 2-ethylphenyl; benzoyl; 1-naphthyl; benzoyl; 4-bromophenyl; 2-methoxyphenyl; m-tolyl; alpha, alpha, alpha-trifluoro-m-tolyl; 3-fluorophenyl; 3-chlorophenyl; 3-bromophenyl; (SCN)C₆H₄—; (propylcyclohexyl)benzyl; (hexylcyclohexyl)benzyl; (octylcyclohexyl)benzyl; 2-methylbenzyl; 2-chlorobenzo; 3-chlorobenzo; 4-chlorobenzo; m-toluoyl; and p-toluoyl.

The ITC can be either isolated from natural sources or prepared by chemical synthesis. Natural sources of ITC include cruciferous vegetables such as horseradish, radishes, onions, mustards, alyssum, candytuft, cabbage, and broccoli (U.S. Pat. Nos. 5,725,895, 5,968,567, and 5,968,505, all of which are incorporated by reference).

Non-limiting examples of ITCs include phenethyl isothiocyanate (PEITC), benzyl isothiocyanate (BITC), sulforaphane (SFN); 2,2-diphenylethyl isothiocyanate (2,2-DPEITC), methyl isothiocyanate; ethyl isothiocyanate; propyl isothiocyanate; isopropyl isothiocyanate; n-butyl isothiocyanate; t-butyl isothiocyanate; s-butyl isothiocyanate; pentyl isothiocyanate; hexyl isothiocyanate; heptyl isothiocyanate; octyl isothiocyanate; nonyl isothiocyanate; decyl isothiocyanate; undecane isothiocyanate; phenyl isothiocyanate; o-tolyl isothiocyanate; 2-fluorophenyl isothiocyanate; 3-fluorophenyl isothiocyanate; 4-fluorophenyl isothiocyanate; 2-nitrophenyl isothiocyanate; 3-nitrophenyl isothiocyanate; 4-nitrophenyl isothiocyanate; 2-chlorophenyl isothiocyanate; 2-bromophenyl isothiocyanate; 3-chlorophenyl isothiocyanate; 3-bromophenyl isothiocyanate; 4-chlorophenyl isothiocyanate; 2,4-dichlorophenyl isothiocyanate; R-(+)-alpha-methylbenzyl isothiocyanate; S-(−)-alpha-methylbenzyl isothiocyanate; 3-isoprenyl-alpha, alpha-dimethylbenzyl isothiocyanate; trans-2-phenylcyclopropyl isothiocyanate; 1,3-bis(isothiocyanatomethyl)-benzene; 1,3-bis(1-isothiocyanato-1-methylethyl)benzene; 2-ethylphenyl isothiocyanate; benzoyl isothiocyanate; 1-naphthyl isothiocyanate; benzoyl isothiocyanate; 4-bromophenyl isothiocyanate; 2-methoxyphenyl isothiocyanate; m-tolyl isothiocyanate; alpha, alpha, alpha-trifluoro-m-tolyl isothiocyanate; 3-fluorophenyl isothiocyanate; 3-chlorophenyl isothiocyanate; 3-bromophenyl isothiocyanate; 1,4-phenylene diisothiocyanate; 1-isothiocyanato-4-(trans-4-propylcyclohexyl)benzene; 1-(trans-4-hexylcyclohexyl)-4-isothiocyanatobenzene; 1-isothiocyanato-4-(trans-4-octylcyclohexyl) benzene; 2-methylbenzyl isothiocyanate; 2-chlorobenzo isothiocyanate; 3-chlorobenzo isothiocyanate; 4-chlorobenzo isothiocyanate; m-toluoyl isothiocyanate; p-toluoyl isothiocyanate and the like. Wang, X., et al., J. Med. Chem., 54(3), 809-816 (2011), which is incorporated by reference, also discloses ITCs can be used within the full scope of the invention.

Any of the ITCs can be conjugated to another entity to form a conjugate. Entities to which the ITCs can be conjugated include but are not limited to any thiol group that can be substituted on the ITC, including but not limited to glutathione, N-acetylcysteine, cysteine, and methionine. The thiol conjugate, while often less potent than the parent compound, is sometimes less toxic and more stable. In specific embodiments, the thiol conjugates of ITC are L-Cys, glutathione, and N-acetyl-L-cysteine conjugates.

Furthermore, the methods of treatment of an abnormal growth herein also relate to coadministering one or more substances in addition to the at least one ITC to the subject. The term “coadminister” indicates that each of at least two compounds is administered during a time frame wherein the respective periods of biological activity or effects overlap. Thus the term includes sequential as well as coextensive administration of the compounds of the present invention. And similar to administering compounds, coadministration of more than one substance can be for therapeutic and/or prophylactic purposes. If more than one substance is coadministered, the routes of administration of the two or more substances need not be the same. The scope of the invention is not limited by the identity of the substance which may be coadministered. For example, PEITC may be coadministered with another ITC or derivative thereof, or even other pharmaceutically active substances, such as but not limited to interleukin-2,5′-fluorouracil, nedaplatin, methotrexate, vinblastine, doxorubicin, carboplatin, paclitaxel (Taxol), cisplatin, 13-cis retinoic acid, pyrazoloacridine, and vinorelbine to name a few. Appropriate amounts in each case will vary with the particular agent, and will be either readily known to those skilled in the art or readily determinable by routine experimentation.

Solid unit dosage forms may be prepared by mixing the compound, salt or derivative of the present invention with a pharmaceutically acceptable carrier and any other desired additives. The mixture is typically mixed until a homogeneous mixture of the compound of the present invention and the carrier and any other desired additives are formed, i.e., until the compound is dispersed evenly throughout the composition. In one particular embodiment, the present invention is formulated as a solid prepared in a capsule form.

Biodegradable polymers for controlling the release of the compound include, but are not limited to, polylactic acid, polyepsilon caprolactone, polyhydroxy butyric acid, polyorthoesters, polyacetals, polydihydro-pyrans, polyanhydrides, polycyanoacrylates, cross-linked or amphipathic block copolymers of hydrogels, cellulosic polymers, and polyacrylates.

For oral administration, the therapeutics can take the form of, for example, tablets or capsules prepared by conventional means with pharmaceutically acceptable excipients such as binding agents, e.g., pregelatinized maize starch, polyvinylpyrrolidone or hydroxypropyl methylcellulose; fillers, e.g., lactose, microcrystalline cellulose or calcium hydrogen phosphate; lubricants, e.g., magnesium stearate, talc or silica; disintegrants, e.g., potato starch or sodium starch glycolate; or wetting agents, e.g., sodium lauryl sulphate. The tablets can be coated by methods well known in the art. The preparations can also contain buffer salts, flavoring, coloring and sweetening agents as appropriate.

The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. As used herein, the term “pharmaceutically acceptable” means approved by a regulatory agency of the Federal or a state government or listed in the U.S. Pharmacopeia or other generally recognized pharmacopeia for use in animals, and more particularly in humans.

Various pharmaceutically acceptable salts, ether derivatives, ester derivatives, acid derivatives, and aqueous solubility altering derivatives of the active compound also are encompassed by the present invention. The present invention further includes all individual enantiomers, diastereomers, racemates, and other isomer of the compound. The invention also includes all polymorphs and solvates, such as hydrates and those formed with organic solvents, of this compound. Such isomers, polymorphs, and solvates may be prepared by methods known in the art, such as by regiospecific and/or enantioselective synthesis and resolution, based on the disclosure provided herein.

Suitable salts of the compound include, but are not limited to, acid addition salts, such as those made with hydrochloric, hydrobromic, hydroiodic, perchloric, sulfuric, nitric, phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; salts made with saccharin; alkali metal salts, such as sodium and potassium salts; alkaline earth metal salts, such as calcium and magnesium salts; and salts formed with organic or inorganic ligands, such as quaternary ammonium salts.

Additional suitable salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydra bamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compound of the present invention.

The present invention includes prodrugs of the compound of the present invention. Prodrugs include, but are not limited to, functional derivatives of isothiocyanates that are readily convertible in vivo into isothiocyanates. Conventional procedures for the selection and preparation of suitable prodrug derivatives are described, for example, in “Design of Prodrugs”, ed. H. Bundgaard, Elsevier, 1985.

In the present invention, the method involves administering to the patient an effective amount of the ITC conjugate in a dosage regimen comprising administering to the patient a dosage form comprising a 20-80 mg capsule, two to three times daily, during the post-initiation phase of lung cancer such that there is intervention after cell commitment to dysplasia. The ITC conjugate can be administered orally.

The dosage regimen (amount and interval) of the compound of the present invention may vary according to a variety of factors such as underlying disease states, the individual's condition, weight, sex and age, the mode and route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yield efficacy without toxicity requires a regimen based on the kinetics of the drug's availability to target sites. This involves a consideration of the absorption, distribution, metabolism, and excretion of a drug.

A medicament comprising an ITC may be prepared by standard pharmaceutical techniques known in the art, depending upon the mode of administration and the particular disease to be treated. The medicament will usually be supplied as part of a sterile, pharmaceutical composition, which will normally include a pharmaceutically acceptable carrier. This pharmaceutical composition may be in any suitable form, (depending upon the desired method of administering it to a subject). It may be provided in unit dosage form, will generally be provided in a sealed container and may be provided as part of a kit, which may include instructions for use and/or a plurality of unit dosage forms.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions can be employed as carriers, particularly for injectable solutions. Suitable pharmaceutical carriers are described in “Remington's Pharmaceutical Sciences” by E. W. Martin, which is incorporated by reference.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions) and lozenges. Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. Specific examples of excipients include but are not limited to poly-ethylene glycol (PEG), dimethyl sulfoxide (DMSO), ethanol and mixtures thereof. For the preparation of suspensions oils (e.g. vegetable oils) may be used to provide oil-in-water or water in oil suspensions. In certain situations, delayed release preparations may be advantageous and compositions which can deliver, for example, PEITC or a derivative or conjugate thereof in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by ionophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists, which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions may include suspending agents and thickening agents. Excipients, which may be used for injectable solutions, include but are not limited to water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. The pharmaceutical compositions may contain preserving agents, solubilising agents, stabilising agents, wetting agents, emulsifiers, sweeteners, colorants, odorants, salts (substances of the present invention may themselves be provided in the form of a pharmaceutically acceptable salt), buffers, coating agents or antioxidants. They may also contain therapeutically active agents in addition to the substance of the present invention.

Dosages of the substance of the present invention can vary between wide limits, depending upon the disease or disorder to be treated, the age and condition of the individual to be treated, etc. and a physician will ultimately determine appropriate dosages to be used.

The invention is also directed towards methods of sensitizing an abnormal tissue to a chemotherapeutic agent. The methods comprise screening at least a portion of an abnormal tissue for at least one mutant form of p53 protein, and administering at least one isothiocyanate (ITC) to the abnormal tissue if the abnormal tissue is positive for the at least one mutant form of p53.

As used herein, “sensitizing a tissue to a chemotherapeutic agent” is used to mean that the abnormal tissue or growth, as defined herein, refers to an increased sensitivity or reduce the resistance of an abnormal growth or tissue, or an organism as a whole, responding to a therapeutic treatment. As used herein, the term “sensitizing.” An increased sensitivity or a reduced sensitivity to a therapeutic treatment is measured according to known methods in the art for particular treatments and methods. Increased sensitivity can be measured by such methdods including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in an organism by measuring, for example, the tumor size reduction over a period of time, for example, 6 months for treatment of a human, and 4-6 weeks for treatment of a mouse. A composition or a method sensitizes a response to a therapeutic treatment if the increase in treatment sensitivity or the reduction in resistance is 5% or more, for example, at least about 5%, at least about 10%, at least about 20%, at least about 30%, at least about 40%, at least about 50%, at least about 60%, at least about 70%, at least about 80%, at least about 90% or more. Another way to measure sensitivity, for example, would be to compare the sensitivities of the abnormal tissue or the same type of abnormal tissue from another individual with and without administration of at least one ITC. A tissue or subject would be sensitized if there is a 1.2 fold difference in sensitivity or more to the therapeutic agent, compared to treatment sensitivity or resistance in the absence of such composition or method. For example, a subject or tissue exhibiting at least about 2-fold increase, at least about 3-fold increase, at least about 4-fold increase, at least about 5-fold increase, at least about 10-fold increase, at least about 15-fold increase, at least about 20-fold increase or more, would be “sensitized” for the purposes of the present invention. The determination of sensitivity or resistance to a therapeutic treatment is routine in the art and within the skill of an ordinarily skilled clinician.

The following examples are meant to be illustrative and not intended to limit the scope of the invention described herein.

### EXAMPLES

Experimental protocols in Example 1 are adapted or reprinted herein with permission from “Selective Depletion of Mutant p53 by Cancer Chemopreventive Isothiocyanates and Their Structure—Activity Relationships,” Xiantao Wang, et al., J. Med. Chem. Copyright (2011) American Chemical Society.”

### Example 1

### Materials and Methods

Human H596, A549, HCT116, MDA-MB-231, MDA-MB-468, MCF-7, DU145, MCF-10A, SW480, and SCC-4 cells were obtained from ATCC (Manassas, Va.). H1299-175H cells were kindly provided by Dr. Maria Laura Avantaggiati (Georgetown University). Cells were maintained in DMEM or RPMI-1640 medium supplemented with 10% FBS (Hyclone). DMSO, PEITC, BITC, SFN, AITC, NMPEA, and monochlorobimane were purchased from Sigma-Aldrich (St. Louis, Mo.). 2,2-Diphenylethyl ITC, 4-phenoxybenzyl ITC, 4-methoxybenzyl ITC, 4-chlorobenzyl ITC, Dα-methylbenzyl ITC, trityl ITC, and 3-phenylpropyl ITC were purchased from Trans World Chemicals (Rockville, Md.). Erucin and 4-phenylbutyl ITC were from LKT Laboratories, Inc. (St. Paul, Minn.). SFN was a generous gift from Dr. Stephen Hecht (University of Minnesota, MN), and 5-phenylpentyl ITC and 6-phenylhexyl ITC were generous gifts from Dr. Arun K. Sharma (Penn State Hershey College of Medicine, PA). All the compounds are with purities ≧95% as determined by gas-liquid chromatography or HPLC. The p53 DO-1 and β-actin antibodies were purchased from Santa Cruz Biotechnology (Santa Cruz, Calif.), horseradish peroxidase-labeled goat antimouse secondary antibodies were purchased from GE-Healthcare (Pittsburgh, Pa.), and Bio-Rad Protein Assay was from Bio-Rad (Hercules, Calif.).

Whole cell lysates were prepared in lysis buffer (20 mM HEPES, pH 7.4, 2 mM EGTA, 50 mM β-glycerol phosphate, 1% Triton X-100, 10% glycerol, 1 mM DTT, 1 mM PMSF, 10 mg/mL leupeptin, 10 mg/mL aprotinin, 1 mM Na₃-VO4, and 5 mM NaF). For immunoblot analysis, equal amounts of protein were resolved on 4-12% NuPAGE BisTris gels (Invitrogen) and transferred to PVDF membranes (Millipore). The membranes were blocked with 5% milk in TBST and were then probed with p53 (DO-1) or β-actin antibody. Proteins were detected using enhanced chemiluminescence (ECL) reagents (GE Healthcare).

Total RNA was extracted from the cells using the RNeasy kit (Qiagen). Reverse transcription PCR was performed using SuperScript One-Step RT-PCR kit (Invitrogen) according to the manufacturer's instructions. Primers for p53 were 5′-TTCTTGCATTCTGGGACAGCC-3′ (sense) and 3′-GGCCTCATTCAGCTCTCGGAAC-5′ (antisense). Primers for β-actin were 5′-TGGGCATGGGTCAGAAGGAT-3′ (sense) and 3′-GAGGCGTACAGGGATAGCAC-5′ (antisense). β-Actin was used as an internal standard for RNA normalization.

BL21 (DE2) Escherichia coli were transformed with pET29b wild-type or mutant (G245C) p53 DNA binding domain (DBD) plasmids. The E. coli were then cultured in LB media supplemented with 100 μM ZnCl₂ and 25 μg/mL kanamycin at 37° C. until OD600=0.6-0.8. Protein expression was induced by 0.1 mM isopropyl β-D-1-thiogalactopyranoside for 16 h at 37 gC for wild-type and 16 gC for mutant p53. Bacterial pellets were then harvested by centrifugation at 5000×g, 10 min, 4 gC, and lysed via sonication in 50 mM Tris, 50 mM KCl, 5 mM dithiothreitol, 1 mM PMSF, pH 7 buffer. The soluble fraction was passed through a Capto S cation exchange column (GE Healthcare) and eluted with 50 mM Tris, 200 mM KCl, 5 mM DTT, pH 7. Eluted protein was dialyzed overnight against a non-salt dialysis buffer (50 mM Tris, 5 mM DTT, pH 7) and loaded onto a HiTrap Heparin HP column (GE Healthcare). The p53 DBD was purified via a AKTApurifier-10 protein liquid chromatography system (GE Healthcare) with an increasing KCl salt gradient, and purity was tested by SDSPAGE and Coomassie blue staining. Purified protein was dialyzed in PBS, and protein concentrations were determined prior to performing fluorescence experiments.

In a white 96-well plate, 100 μL of purified mutant (G245C) p53 DNA-binding domain protein in PBS was treated with DMSO, 100 μM ITCs in DMSO, or 500 μM iodoacetamide (positive binding control). An additional well containing 100 μL of PBS was used to determine background. Samples were incubated with compounds in the dark for 1 h at 32 gC, after which 5 μA of 10 mM monochlorobimane was added (in the dark), and samples were further incubated in the dark at 32 gC for 30 min. Fluorescence readings were then obtained using a BIO-TEK Synergy HT plate reader (λex 380 nm; λem 485 nm).

A SPEX FluoroMax-2 spectrofluorometer was used to collect emission spectra: λex 280 nm; λem 285-400 nm; integration time 0.5 s/nm; three scans averaged; slit width 5 nm. Data was collected after 6 μM of mutant (G245C) p53 DBD (2504) was incubated for 1 h at 32 gC alone or with DMSO or 60 μM ITCs.

Caspase-3 activity was measured by detection of the cleavage of a colorimetric caspase-3 substrate, N-acetyl-Asp-Glu-Val-Asp (DEVD)-p-nitroaniline, using an assay kit (R&D Systems, Inc., Minneapolis, Minn.). The assay was performed according to the instructions of the manufacturer. In brief, cells were treated with drugs for 24 h. The cells were collected and lysed in ice-cold lysis buffer provided by the manufacturer. The same amount of protein extracts (100-200 μg) were incubated in a reaction buffer containing N-acetyl-DEVD-p-nitroaniline at 37 gC for 2-4 h. The levels of the resulting proteolytic fragment p-nitroanilide were measured as optical density at 405 nm with a plate reader. The data represent the mean±SD of three independent experiments.

MDA-MB-231 breast cancer cells were treated at 40-50% confluency with DMSO or 10 μM BITC, 2,2-diphenylethyl ITC, 4-methoxybenzyl ITC, trityl ITC, cyclohexylmethyl ITC, SFN, 3-PPITC, or 4-PBITC in DMSO for 24 h, after which time cells were collected, washed twice with cold PBS, and resuspended in 1× binding buffer from FITC-annexinVapoptosis kit (BD Pharmingen). Then, 100 μL of each sample was added to a 5 mm tube, and the annexin V apoptosis kit protocol was followed. Samples were immediately analyzed with flow cytometry.

### Example 1

### Results

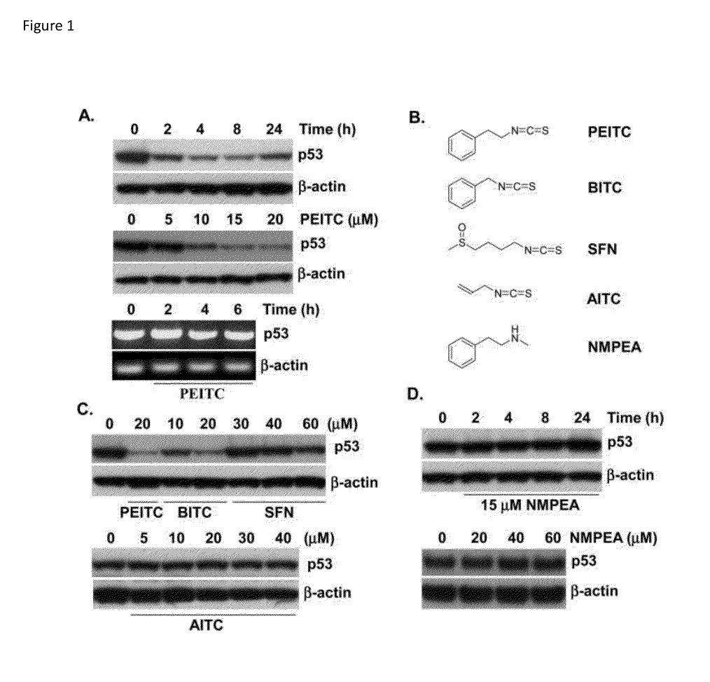

Treatment of human H596 (G245C) non-small-cell lung cancer cells with 15 μM PEITC caused a rapid depletion of mutant p53 protein. Depletion occurred as early as 2 h and peaked at 4 h; it was sustained during 24 h treatment and was concentration-dependent (FIG. 1A). The expression of other proteins, including Bax, DRS, Hsp90, and JNK, was not altered (not shown). To determine whether depletion occurs at the post-transcriptional or transcriptional level, reverse transcription polymerase chain reaction (PCR) was performed on RNA derived from DMSO- and PEITC treated H596 cells. As shown in FIG. 1A (lower panel), 2, 4, and 6 h after PEITC treatment, the mutant p53 mRNA levels remained unchanged. Therefore, PEITC depleted mutant p53 without causing changes in the p53 mRNA expression levels. These results strongly suggest that PEITC depletes mutant p53 at the post-transcriptional level. The effects of other widely studied naturally occurring ITCs, benzyl ITC (BITC), SFN, and AITC, was examined on mutant p53 protein levels. H596 cells were incubated with PEITC, BITC, SFN, or AITC for 2 h. FIG. 1 shows that BITC at 10 μM significantly reduced the level of mutant p53, while at 20 μM, it showed a similar potency to PEITC. To further investigate the structural requirement of ITCs to deplete mutant p53, N-methylphenethylamine (NMPEA), an analog of PEITC lacking an ITC functional group, was used. FIG. 1 also shows that NMPEA at 15 μM failed to alter the mutant p53 protein level even up to 24 h or at 60 μM for 2 h. Because the ITC functional group is highly electrophilic, these results indicate that the ITC group is essential for the effect, possibly through binding to target proteins. Moreover, the structure of the side-chain moiety appears to dictate the potency of mutant p53 depletion by ITCs.

Depletion of mutant p53 in other cells was examined. Various tumor cells with mutant p53 were used, including MDA-MB-231 (breast cancer, R280K), MDA-MB-468 (breast cancer, R273H), DU145 (prostate cancer, P274L/V223F), SW480 (colon cancer, R273H), and SCC-4 (oral cancer, P151S). Notably, while the types of mutations among the cell lines vary, all are located in the core DNA-binding domain.

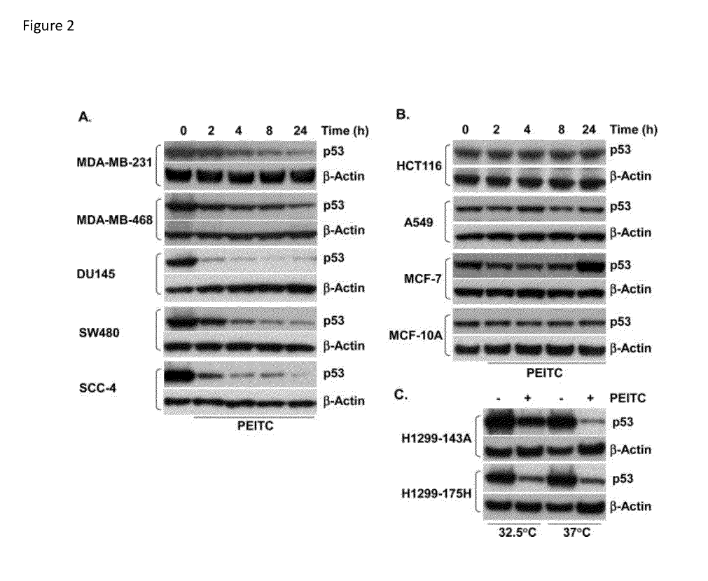

The five cell lines were treated with 10 or 15 μM PEITC, depending on the observed cytotoxicity. FIG. 2 shows that PEITC depleted mutant p53 in all five cell lines in a time-dependent manner, and certain cells appeared more responsive than others. These results clearly show that the effects of PEITC on mutant p53 are not specific to a certain p53 mutation.

To determine whether wild-type p53 is affected by PEITC treatment, A549 lung cancer cells, HCT116 colon cancer cells, MCF-7 breast cancer cells, and MCF-10A human normal mammary epithelial cells, all of which express wild-type p53, were used. Cell lines were treated with 15 or 20 μM PEITC for 2, 4, 8, and 24 h. Contrary to mutant p53, wild-type p53 expression did not decrease over the treatment period.

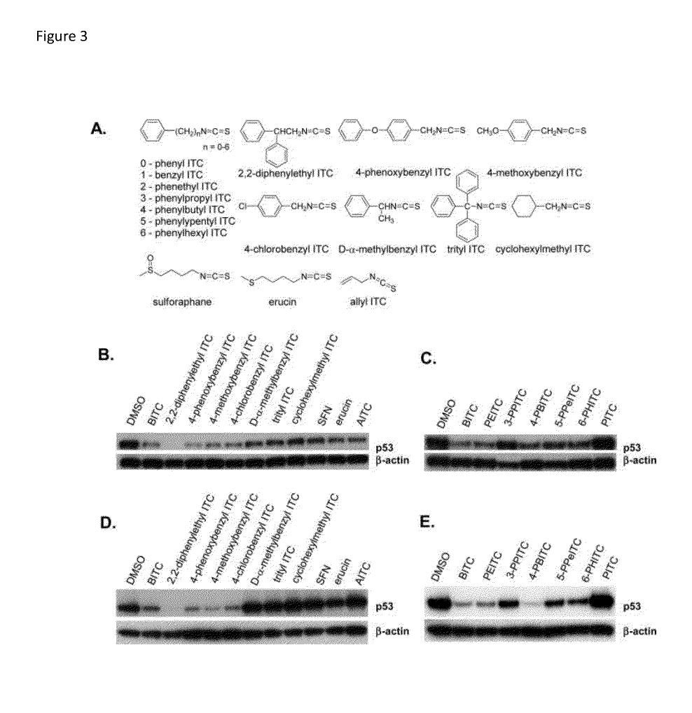

To investigate whether covalent binding by ITCs is a trigger of mutant p53 depletion, ITCs with bulky substituent(s) adjacent to the —N═C═S to create steric hindrance, including D-α-methylbenzyl ITC and trityl ITC were selected for use. Aromatic ITCs with varying alkyl chain length from phenyl ITC to 6-phenylhexyl ITC (6-PHITC) were used. Structures of the ITCs are shown in FIG. 3A. The aliphatic compounds used include cyclohexylmethyl ITC, SFN, erucin, and AITC. As shown in FIG. 3B-E, mutant p53 is depleted similarly in H596 (FIGS. 3B,C) and MDA-MB-231 (FIGS. 3D, E) cancer cells treated with 20 or 10 μM ITCs, respectively, for 24 h. Also, at equimolar concentrations, BITC and PEITC deplete mutant p53 to approximately the same extent, while SFN depletes p53 significantly less in H596 or no effect in MDMBA cells, compared with DMSO control.

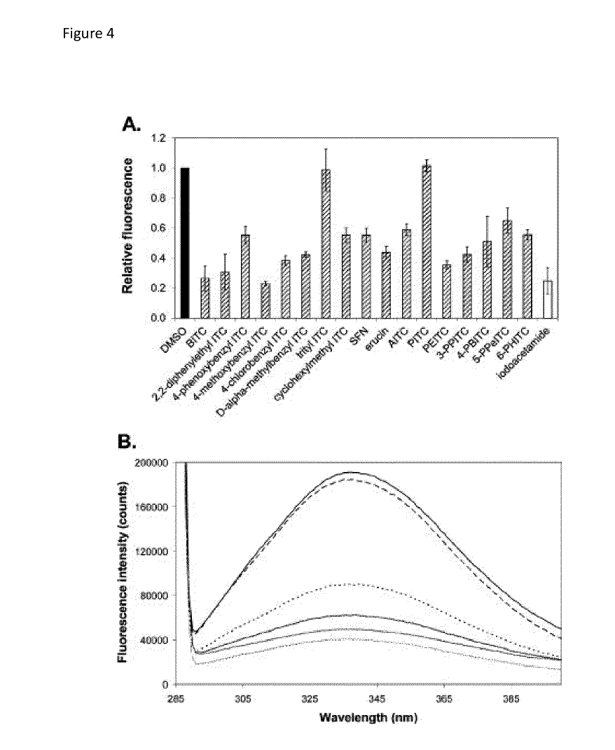

A monochlorobimane fluorometric assay was used to study cysteine modification of mutant (G245C) p53 DBD by ITCs and compared intrinsic tryptophan fluorescence intensities as an indicator of conformational changes. The G245C point mutation is especially relevant because it is the p53 mutation present in the H596 non-small-cell lung cancer cell line studied here. FIG. 4A shows the relative amount of free thiols present, as indicated by relative fluorescence intensities, in the mutant (G245C) p53 DBD after 1 h incubation with DMSO (negative control), ITCs, or iodoacetamide (positive control). BITC, 2,2-diphenylethyl ITC, 4-methoxybenzyl ITC, 4-chlorobenzyl ITC, PEITC, and 3-PPITC show greater binding affinities for mutant (G245C) p53 DBD than do trityl ITC, cyclohexylmethyl ITC, SFN, AITC, and PITC (p<0.05). These affinities correlate well with their respective activity to deplete mutant p53.

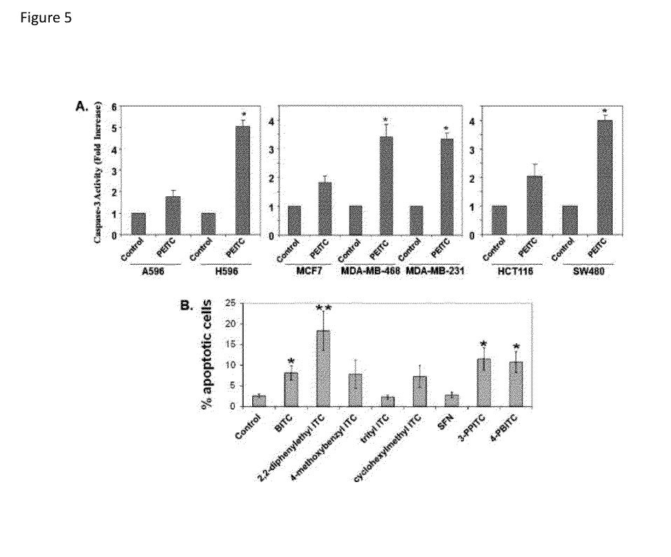

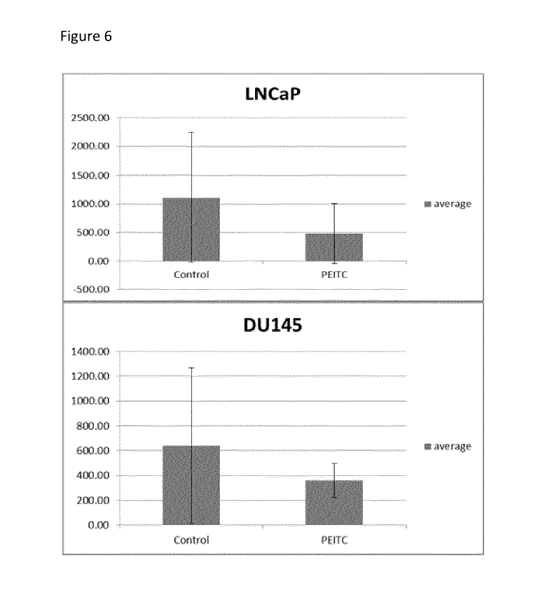

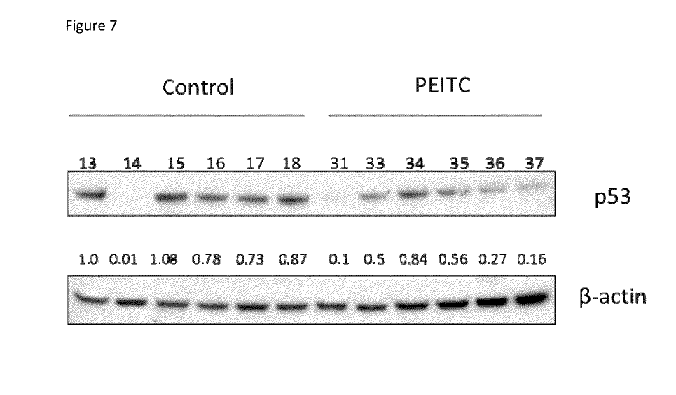

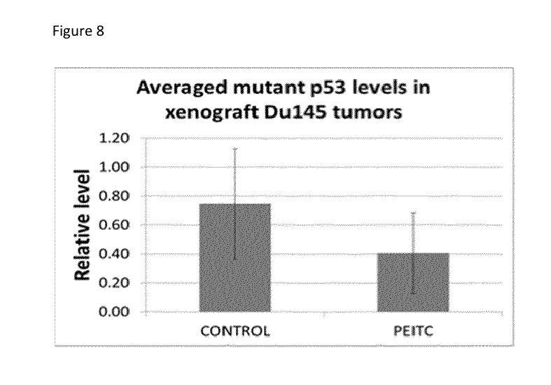

The role of p53 in the induction of apoptosis by ITCs is not well established and likely to be cell-specific. Mutant p53 renders cancer cells resistance to chemotherapeutic drugs; if ITCs can deplete mutant p53, then ITCs should be more cytotoxic in cells with mutant p53 protein. To examine this possibility, we assessed the cytotoxicity induced by PEITC in cancer cells containing either wild-type or mutant p53. Three sets of cell lines from lung, breast, and colon were used: A549 (wild-type p53) and H596 (mutant p53) lung cancer cells, MCF-7 (wild-type p53) and MDA-MB-231 and MDA-MB-468 (mutant p53) breast cancer cells, and HCT116 (wild-type p53) and SW480 (mutant p53) colon cancer cells. After treating with PEITC for 24 h, apoptosis was determined by caspase-3 activity assay. As shown in FIG. 5A, all the mutant p53 cancer cells appeared significantly more sensitive than the wild-type cells to PEITC-induced apoptosis.

To examine the correlation between depletion of mutant p53 and apoptosis by ITCs, FITC-annexin V staining assay was used to quantify apoptotic cells. MDA-MB-231 cancer cells were treated with either DMSO or 10 μM of various ITCs for 24 h; FIG. 5B shows that treatment with BITC, 2,2-diphenylethyl ITC, 3-PPITC, or 4-PBITC, which all deplete mutant p53 (FIG. 3B-E), results in a greater induction of apoptosis than treatment with DMSO (p<0.05). 2,2-Diphenylethyl ITC, the most potent depletors of mutant p53 studied, is the strongest inducer of apoptosis (p<0.05 compared with BITC).

### Example 2

To answer the questions whether wild-type and mutant p53 can be depleted by PEITC treatment, and whether mutant p53 depletion, which was observed in vitro, can be observed in vivo, a xenograft model was employed using two prostate cancer cell lines, LNCap (wild-type p53) and DU145 (mutant p53). Briefly, 30 BALB/C nu/nu transgenic mice (purchased from Charles River) were randomized into two groups, and fed with powder diet AlN93 (purchased from Research Diets, Inc.) with or without addition of 5 μM PEITC compound (purchased from Sigma-Aldrich). After two weeks of feeding, two xenograft tumors each carrying 106 cells in a volume of 100 μL were established by injection into both flanks of a mouse. Mice were continuously fed with diets with or without PEITC for additional 8 weeks before sacrifice. Diet intake and tumor volume were measured twice every week. Tumors were harvested at the end of bioassay for immunohistochemistry and immunoblot studies. Results showed that the tumor volumes of both LNCap and DU145 were significantly decreased in the PEITC-treated animals, compared with that on a diet without PEITC. The p53 level detected by immunoblot showed a moderate, yet significant, decrease in DU145 tumors, but not in LNCap tumors. Immunohistochemistry assays were carried out to examine the apoptotic markers, such as TUNEL, and anti-proliferation marker Ki67 in tumors from the PEITC-treated vs. untreated mice. This study provides the first in vivo evidence that mutant p53 can be depleted in tumors of mice fed PEITC-containing diet and its depletion seems to correlate with growth inhibition in the mutant p53 tumors.

### Example 3

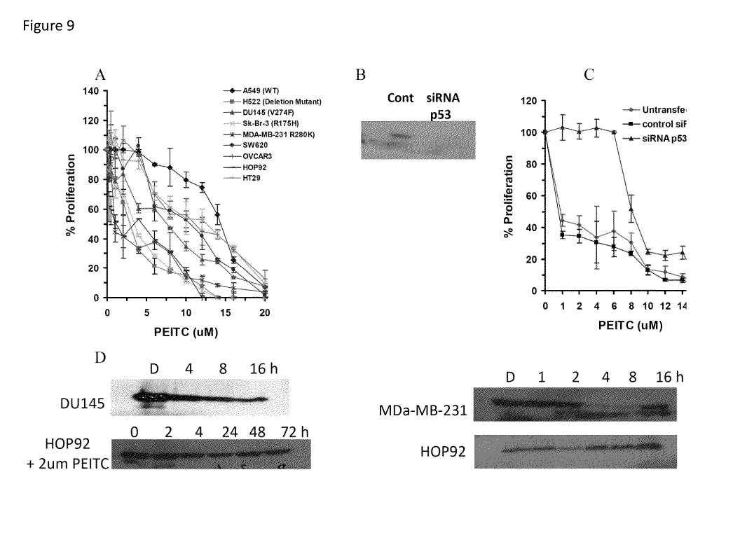

The previous experiments showed that PEITC exposure caused a marked reduction in the level of mutant p53 protein in a variety of tumor cell lines at a concentration of ≧10 uM, whereas no significant decrease was observed for the wild type (WT) p53 protein. Next, the effect of PEITC exposure on tumor growth of the tumor cell lines expressing mutant or the WT p53 protein was examined. Tumor cell lines expressing mutant p53 protein showed greater inhibition of cell proliferation at lower doses of PEITC compared with WT p53 cells (FIG. 9A). HOP-92 (R175L) cell line showed 40% inhibition of cell proliferation at dose of 250 nM and mda-mb-231 (R280K) showed 55% inhibition at a dose of 1 μM. Cell line expressing WT p53 (A549) showed a 25% and 45% reduction in cell proliferation at dose of 12 and 14 μM respectively, suggesting that mutant p53 cell lines were several fold more sensitive to PEITC exposure. This hypersensitivity to PEITC being mediated through mutant p53 was next examined. The effect of PEITC on mda-mb-231 cells depleted of mutant p53 by >90% was compared to non-specific (NS) siRNA transfected cells as a control (FIG. 9B). Mutant p53 depleted mda-mb-231 cells showed a significant reduction in the sensitivity to cell growth inhibition (FIG. 9C), whereas control siRNA transfected cells were highly sensitive to PEITC. This suggests that the PEITC mechanism is at least partially dependent on mutant p53 protein. Further the effect of PEITC treatment on the level of mutant p53 protein at lower doses was analyzed by western blot analysis (FIG. 9D).

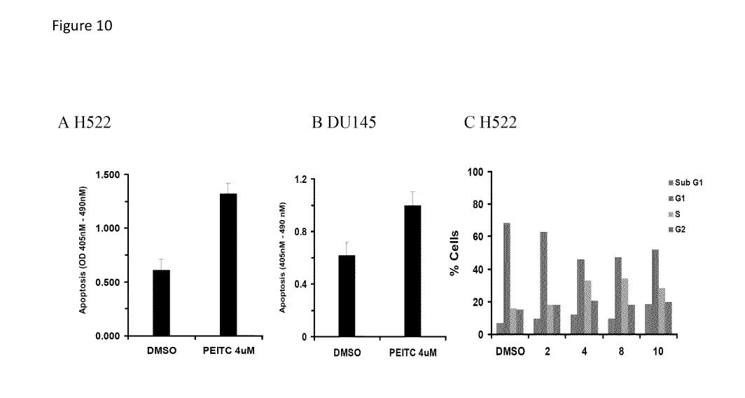

The negative effects of PEITC on cell proliferation might affect the apoptotic potential of the cells and the cell cycle progression. H522 and DU145 cells exposed to 4 uM PEITC showed a two fold increase in apoptosis (FIGS. 10A,B) and S-phase arrest as compared to DMSO treated cells (FIG. 10C).

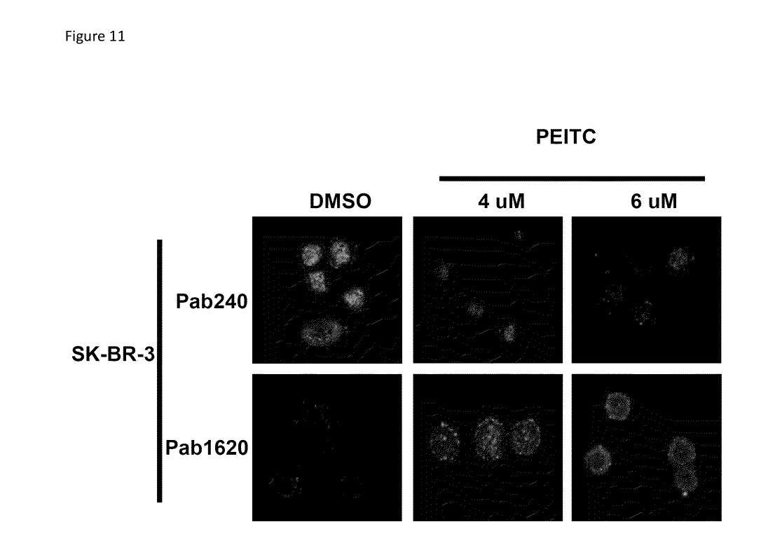

To determine if the induction of apoptosis is due to the restoration of WT conformation to the mutant p53 protein, immunoflourescence analysis was performed. SK-BR-3 cells expressing p53R175H mutant protein were treated with PEITC or DMSO as a control for 4 hrs. Cells were stained with the conformation specific antibody (PAB240) that recognizes the mutant p53 or antibody specific for the WT p53 protein (PAB1620). PEITC treatment induced conformation change in the p53R175H mutant to structure that was recognized by PAB1620, whereas no staining for WT p53 protein was observed in the control DMSO treated cells (FIG. 11).

What is claimed is:

1. A method of treating an abnormal tissue in a subject, the method comprising
a) screening at least a portion of the abnormal tissue for at least one mutant form of p53 protein, and
b) administering at least one isothiocyanate (ITC) to the subject if the abnormal tissue is positive for the at least one mutant form of p53.

2. The method of claim 1, wherein the subject is a mammal.

3. The method of claim 1, wherein the abnormal tissue is cancerous.

4. The method of claim 1, wherein the ITC is sulforophane (SFN), phenylethyl ITC (PEITC), benzyl ITC (BITC).

5. The method of claim 1, wherein the ITC is conjugated.

6. The method of claim 5, wherein the conjugate to the ITC is a thiol conjugate.

7. The method of claim 1, wherein the abnormal tissue is lung tissue, breast tissue, colon tissue and prostate tissue.

8. The method of claim 1, wherein ITC is administered orally.

9. The method of claim 1, further comprising co-administering an anti-cancer agent with the ITC.

10. The method of claim 1, wherein the mutant p53 has a mutation at V143, P151, R175, G245, R248, R249, R273, P274, R280 and R282 of the amino acid sequence of SEQ ID NO:1.

11. A method of altering the three-dimensional conformation of a mutant p53 protein, the method comprising administering at least one isothiocyanate (ITC) to the mutant p53 protein to alter the three-dimensional conformation of the mutant p53 protein.

12. The method of claim 11, wherein the mutant p53 protein is present in a cell or group of cells and the administration of the at least one ITC to the mutant p53 protein comprises administering the ITC to the cell or group of cells.

13. The method of claim 12, wherein the cell or group of cells is comprised within a subject and the administration of the at least one ITC to the mutant p53 protein comprises administering the ITC to the cell or group of cells comprises administration of the at least one ITC to the mutant p53 protein comprises administering the ITC to the subject.

14. The method of claim 13, wherein the subject is a mammal.

15. The method of claim 12, wherein the cell is a cancer cell.

16. The method of claim 11, wherein the ITC is sulforophane (SFN), phenylethyl ITC (PEITC), benzyl ITC (BITC).

17. The method of claim 11, wherein the ITC is conjugated.

18. The method of claim 17, wherein the conjugate to the ITC is a thiol conjugate.

19. The method of claim 15, wherein the cancel cell is a lung cancer cell, a breast cancer cell, a colon cancer cell or a prostate cancer cell.

20. The method of claim 14, wherein ITC is administered orally.

21. The method of claim 14, further comprising co-administering an anti-cancer agent with the ITC.

22. The method of claim 11, wherein the mutant p53 has a mutation at V143, P151, R175, G245, R248, R249, R273, P274, R280 and R282 of the amino acid sequence of SEQ ID NO:1.
